(19) United States
(12) Patent Application Publication
(10) Pub. No.: US 2012/0065716 A1
(43) Pub. Date: Mar. 15, 2012

(54) PORTABLE BODY WARMING DEVICE

(76) Inventors: BRIJESH S. GILL, Houston, TX (US); Charles S. Cox, JR., Bellaire, TX (US); Kevin R. Aroom, Houston, TX (US); Jeffery J. Sheldon, League City, TX (US); Todd L. Westerbeck, League City, TX (US)

(21) Appl. No.: 13/231,018

(22) Filed: Sep. 13, 2011

Related U.S. Application Data
(60) Provisional application No. 61/382,799, filed on Sep. 14, 2010.

(51) Int. Cl. A61F 7/08

(52) U.S. Cl. 607/107

(57) ABSTRACT

A portable body warming device comprised of a housing (1), a powered blower (19), a combustion chamber (25), an activator (27), a heat exchanger (35) within the housing, a portable hydrocarbon gas fuel source (39) and a battery for powering the blower and activator. The activator initiates combustion of fuel gas in the combustion chamber, and an exhaust vent port discharges combustion byproducts from the housing to serve as the portable warmer.

### CROSS-REFERENCE TO RELATED APPLICATION

This application claims the priority of U.S. Provisional Application No. 61/382,799 filed on Sep. 14, 2010, the disclosure of which is incorporated herein by reference for all purposes.

### FIELD OF THE INVENTION

The present invention relates to devices for warming a human body, and particularly relates to a portable air warming device for counteracting hypothermia.

### BACKGROUND OF THE INVENTION

Normal body temperature in humans is 36.5-37.5° C. (98-100° F.). Hypothermia is a condition in which core temperature drops below that required for normal metabolism and body functions, which is defined as a body temperature of below 35.0° C. or 95.0° F. Hypothermia can be mild (32-35° C.), moderate (28-32° C.), severe (20-28° C.), or profound hypothermia (<20° C.).

The aggressiveness of the therapy applied to treat hypothermia depends upon the degree of hypothermia. Treatments range from noninvasive, passive warming which involves the use of a person's own heat generating ability through the provision of properly insulated dry clothing and moving to a warm environment.

The next level of therapy involves active external re-warming which involves applying an external warming source, such as placing the patient in a tub with hot water (of 44° C.), while placing their extremities (arms and legs) outside of the tub, placing hot water bottles in both armpits or warmed forced air (such as those described below).

Hypothermia can result from exposure to a cold environment, such as experienced by those who spend time in very cold climate, such as but not limited to, alpine and ice climbers, polar explorers and scientists, and those who participate in alpine warfare. Additionally, surgical and trauma patients frequently suffer serious complications due to hypothermia.

It is estimated that annually, in the U.S. alone, millions of patients suffer hypothermia during surgical procedures as a result, for example, of loss of blood, shock, exposure to anesthesia, air conditioning, infusion of cold blood, intravenous (IV) solutions, and/or irrigation fluids. Hypothermia also has a negative effect on patients with major injuries. Cardiac output is compromised and enzyme systems become less efficient with the falling body temperature. Hypothermia also exacerbates hemorrhagic shock in multiple ways. The onset of coagulopathy which accompanies hypothermia has been shown to result from malfunction of both clotting factors and platelets.

Although profound hypothermia can be tolerated by immersion or cardiac surgery patients, the presence of hypothermia in trauma patients predicts significantly higher mortality. Mortality doubles for heterogeneous groups of trauma patients at 34° C., and survival after major trauma is rare when the core temperature falls below 32° C. The more severe the injury the greater the negative influence of hypothermia severely on survival.

Similarly those injured, for example, on the battlefield, may suffer hypothermia due to many sources including loss of blood, shock, or exposure during transport in a helicopter as body heat is convectively lost to the environment. This effect is only made worse by significant blood loss or the presence of large surface area burns. To worsen the situation, the body looses central thermoregulation and the ability to shiver and generate body heat after traumatic injury and less body heat is produced peripherally as perfusion decreases due to shock.

The administration of intravenous fluids, often used to treat trauma patients, can itself induce or exacerbate hypothermia if the fluid is not warmed prior to administration, inducing coagulopathy in injured patients, particularly in the presence of acidosis. The condition worsens when the more severely injured patients, who require the most fluids, also have the least ability to tolerate the additional insult of decreased core temperature. To avoid this crisis, fluids may be warmed to normal body temperature. Methods of doing this, particularly in the field, include, for example, using a device such as the portable fluid warming system described in U.S. Pat. No. 7,261,557, WO 2009/018025, the Thermal Angel® (see for example, U.S. Pat. Nos. 6,142,974 and 6,139,528) or the Ranger® blood and fluid warming system (Arizant Inc., Eden Prairie, Minn., USA).

In field hospitals, where there is a ready source of power, treatment for trauma associated hypothermia leads to the adaptation of devices that generate therapeutic warming using convective devices that generate hot air.

Early on these devices were constructed by adapting readily available components that were designed and intended for completely different purposes. For example, a hair dryer or similar source has been used to blow hot air into a container, such as a cardboard box or body bag, in which at least a portion of the patient was contained. These devices all made ready use of duct tape (100 mph tape). Some of these technologies were even adaptable for use in helicopter transport by tapping into the electrical system of the helicopter to energize the heat sources or in the field if a generator were present.

Although portable hair dryers are known to the art (see, for example, U.S. Pat. Nos. 5,155,925; 5,857,262; and 6,959,707), hair dryers are not designed to generate a constant source of warm air and they run hot, risking fire and patient burns, particularly if left unattended. A crude attempt to regulate, more or less, the temperature inside the container requires the use of an on-off cycle by an attentive operator. Furthermore, such devices also have the disadvantage of being dangerous if used as a source of heat in a closed environment due to the potentially toxic exhaust gases mixing with the driven airflow.

For use in, for example, surgical applications and where a ready source of alternating current electricity is available, more sophisticated convective devices has been devised, some of which are described in U.S. Pat. Nos. 7,014,431; 6,876,884; and Publication Nos. US2005/0015127; US2005/0143796; 2006/0122671; US2006/0122672; and US2006/0184215) for warming that generates warm air that is directed for example into thermal blankets (see U.S. Pat. Nos. 4,572,188; 5,405,371; 6,524,332) and inflatable covers (described in U.S. Pat. No. 7,578,837) and adapted clinical garments (WO 2003/086500; US2006/0122671, US2007/0093885). Commercially available devices include the Bair Paws® System and Bair Hugger® Therapy thermal blankets, such as intraoperative, underbody, pediatric, cardiac, and outpatient blankets and related forced-air warming devices, such as the Bair Hugger Model 505, the Model 750, Model 775 all of which operate on alternating current electrical source of power (Arizant Inc., Eden Prairie, Minn., USA). While these systems are commonly used to prevent and treat hypothermia, none are easily portable and can be carried by a medic or a mountaineer. None are stand alone systems, designed for use in areas lacking a readily available alternating current electrical power source, let alone the austere alpine environment or that of the battlefield.

The present invention provides a safe and effective portable convective air warming system for use in therapeutic warming of air to be applied to prevent or treat hypothermia in a patient. It overcomes the limitations of prior technologies by supplying a light, compact, and thus a portable system such that one man or woman can carry it into the field. It is also self contained and is not dependent upon sources of alternating current electrical energy. This device is particularly safe in that it maintains a separation of the air used for combustion and exhaust from that which is warmed and directed towards the patient.

The disadvantages of the prior art are overcome by the present invention, an improved portable body warming device is hereinafter disclosed.

### SUMMARY OF THE INVENTION

The present invention is directed to a portable convective and conductive system for use in therapeutic warming of air to be applied to prevent or treat hypothermia in a patient. In some embodiments, the patient is being treated for trauma. The invention functions as a thermal transfer system such that a heat exchanger takes the heat resulting from the hydrocarbon combustion process and transfers this heat to an air flow which is delivered to the patient, while avoiding the mixing of potentially toxic combustive gases with the warmed therapeutic air stream that is directed at the patient.

The body warming device comprises a combustion system that provides energy in the form of heat, and a blower providing adequate airflow to drive air through a thermal transfer system such as a heat exchanger to capture the heat and deliver it to an outlet intended to be placed in proximity of a patient with or in danger of developing hypothermia.

These and further features and advantages of the present invention will become apparent from the following detailed description, wherein reference is made to the figures in the accompanying drawings.

### BRIEF DESCRIPTION OF THE DRAWINGS

FIG. 1 is an exploded view of an embodiment of the present invention.

FIG. 2 is a pictorial view of the embodiment shown in FIG. 1.

FIG. 3 is a top view of the embodiment shown in FIG. 2.

FIG. 4 is a front view of the embodiment shown in FIG. 2.

FIG. 5 is a pictorial view of a portable body warmer being used in one embodiment to deliver warmed air through a flexible conduit to an enclosure containing a patient at risk of hypothermia.

FIG. 6 is an exploded view of another embodiment of the invention.

FIG. 7 is an exploded view of some of the fuel system components shown in FIG. 6.

FIG. 7A is a pictorial view of the assembled fuel system components in FIG. 7.

FIG. 8 is an exploded view of some of the components shown in FIG. 6.

FIG. 8A is a pictorial view of the assembled components in FIG. 8.

FIG. 9 is an exploded view of heat exchanger components.

FIG. 9A is a pictorial view of the assembled components in FIG. 9.

FIG. 10 is an exploded view of another embodiment of the invention.

FIG. 10A is a pictorial view of the assembled components in FIG. 10.

### DETAILED DESCRIPTION OF PREFERRED EMBODIMENTS

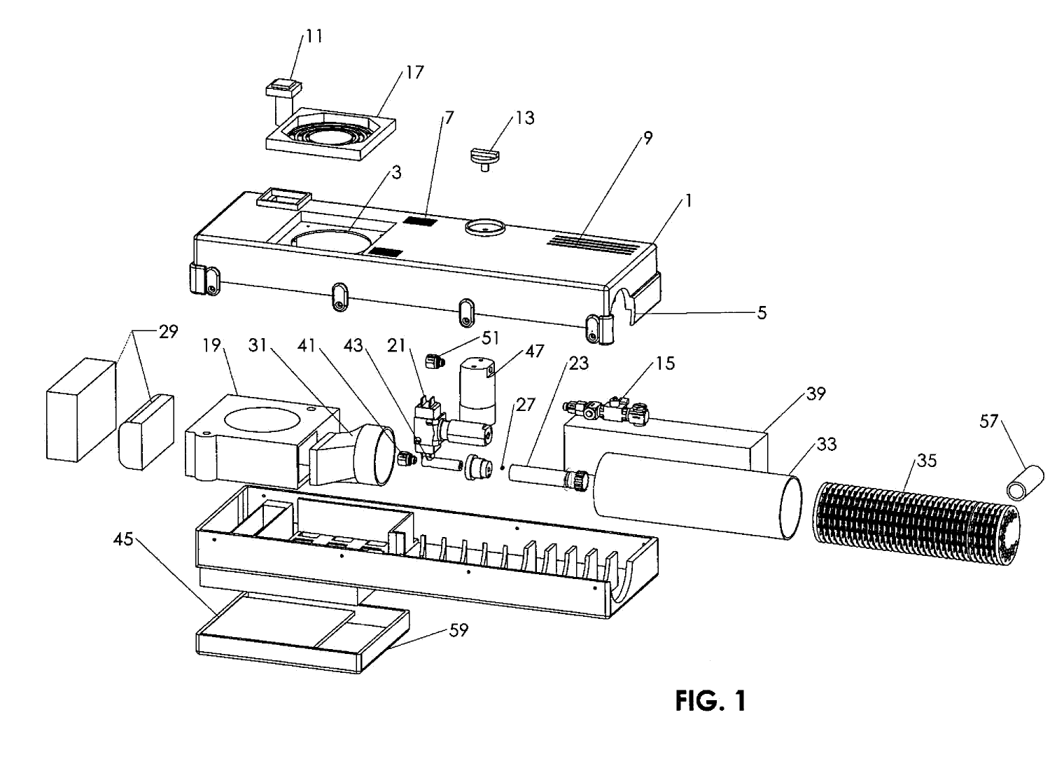

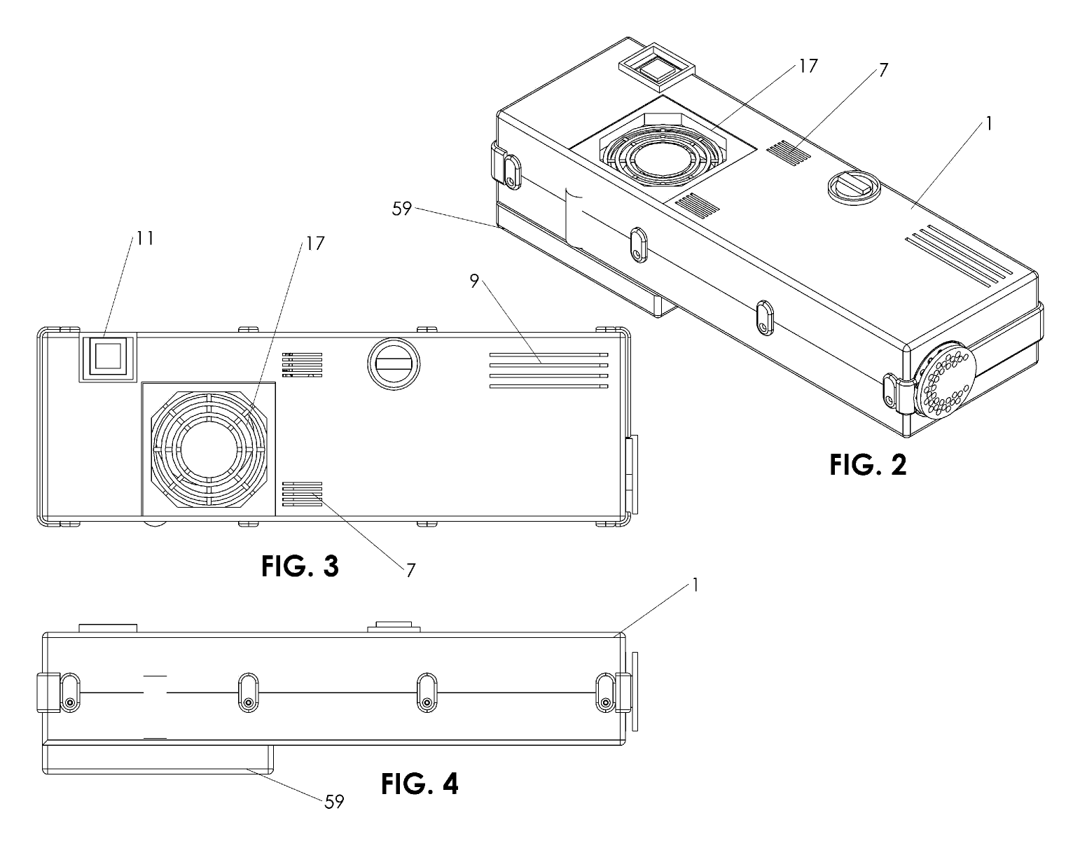

One embodiment of the present invention is directed toward a portable body warmer comprising an outer housing 1 designed to be light but strong. Such an outer housing can be constructed of, but is not limited to, plastic, aluminum, titanium and carbon fiber. In another preferred embodiment, the housing 1 is made of plastic. In one embodiment, the outer housing 1 is rectangular. In this embodiment, the dimensions of a rectangular housing 1 is no more than 30 centimeters×11 centimeters×8 centimeters. The rectangular housing 1 comprises a top surface, a bottom surface and two side surfaces (FIG. 1). In this embodiment, the top surface of the housing 1 is no more than 30 centimeters×11 centimeters and comprises multiple openings for air intake and discharge. In a preferred embodiment, the dimensions of a rectangular housing 1 is 24 centimeters×11 centimeters×5 centimeters.

In a preferred embodiment, an air inlet port 3 receives ambient air and is in fluid communication with an air discharge port 5 through which therapeutic warmed air passes on the way to the patient in need of warming. In one embodiment, the housing 1 also comprises an intake port 7 which allows air for combustion to enter the device. Alternatively, air for combustion may be drawn through intake port 7 by the blower 19.

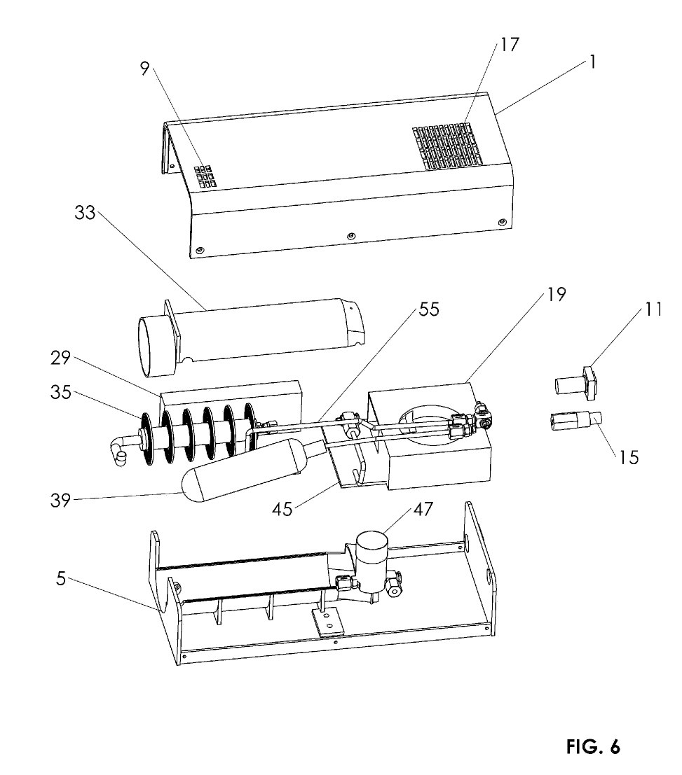

In an alternative embodiment, as illustrated in FIG. 6, the air to be used for combustion and the air to be warmed for therapy enters the device through the same opening and some of the air is forced by the fan-blower 19 through a cowling 31 that connects the fan-blower 19 to a generally cylindrical tube 33 within which lies a heat exchanger 35 through which thermal contact with the combustion chamber tube 25 where by the therapeutic air is warmed. In this embodiment the fan-blower 19 also blows some of the air into the combustion pathway. To assure that there is no mixing of air streams in the device, in some embodiments, a shroud is located between the fan-blower 19 and the intake opening. In other embodiments, a divider is inserted.

In the FIG. 6 embodiment, all air for proper operation enters through a single inlet port that can contain a filter 17 and there is a exhaust vent 9 through which combustive gases are released. Thus, the top of housing contains 2 openings. In preferred embodiments the inlet port and the exhaust vent 9 are located at opposite ends of the housing 1. In this embodiment, the discharge port 5 is in the side of the housing.

All air for proper operation enters through inlet port 3 and there is a exhaust vent 9 through which combustive gases are released. In preferred embodiments, the inlet port 3 and the exhaust vent 9 are located at opposite ends of the housing 1. In this embodiment, the discharge port 5 is in the side of the housing.

In one embodiment, the top surface of the housing 1 is no more than 30 centimeters×10 centimeters and comprises multiple openings.

In some embodiments, there is a first opening in the housing for a latching power switch 11, which powers up the electrical circuits in the device when “on” or engaged; a second opening for a manually actuated master cutoff valve knob 13 which connects to a manual cutoff valve 15 which allows the fuel gas to flow into the gas delivery line when the manually actuated master cutoff valve knob 13 is turned to the on position from the off position; a third, air inlet port 3 such that a filter 17 can be placed exterior of a fan-blower 19, through this air inlet port 3 exterior ambient air is drawn by a fan-blower 19 through the filter 17 and passes through the device to an air discharge port 5 through which therapeutic warmed air passes out of the device; and a fourth opening, serving as an exhaust vent port 9 through which combustive gases are released. The air inlet port 3 for the intake of ambient air and the exhaust vent port 9 for the release of the combustive gases are preferably located at opposite ends of the housing 1 to prevent exhaustive gases from contaminating the exterior air that is being drawn in and used for the therapeutic air stream and is directed at the patient. In some embodiments, the air filter 17 may be held in place over the air inlet port 3 between the fan blower and the exterior by a filter cover.

In one embodiment, the manually actuated master cutoff valve knob 13 is turned to the on position from the off position which opens the manual cutoff valve 15 and allows the fuel gas to flow into the gas delivery line when the latching power switch 11 is engaged to power up the electrical circuits in the device. The use of a two switch system reduces the possibility of accidental ignition and thus preserves both gas and battery life because it requires the user to open the master cutoff valve 15 and press the power switch 11 to turn on the device. In another embodiment, the master cutoff valve knob 13 and the latching power switch 11 can be operated at the same time, more or less simultaneously.

In one embodiment, distal the manual cutoff valve 15, the gas delivery line also contains a pressure switch 21 which in the absence of the correct fuel gas pressure opens the electrical circuit and prevents the device from functioning, in doing so, among other things, this enhances safety and preserves battery life and prevents activation of combustion in the absence of combustible fuel. Preferably, the master cutoff valve 15 would be opened just prior to pressing the latching power switch 11.

The present invention uses heat from hydrocarbon combustion preferably hydrocarbon combustion that takes place in the absence of an open flame. As an example, in one embodiment, the present invention may be used with a gaseous hydrocarbon such as butane which is allowed to flow onto a catalytic substrate 23, such as a platinum mesh, and then combust. The fuel, for example butane, combines with oxygen and liberates heat which then heats the platinum mesh. In this embodiment, the temperature of the mesh stabilizes at the combustion temperature of the fuel, for example butane, thereby allowing combustion to occur on the surface of the platinum mesh. In a preferred embodiment, the air which is heated and applied to the patient is completely isolated from the air which is used for combustion to generate heat and which contains combustive gases. To accomplish this, combustion occurs within a sealed system comprising combustion chamber tube 25.

In a preferred embodiment, within this combustion chamber tube 25 is a catalyst substrate 23 where the catalytic combustion of gaseous fuel-air mixture occurs. In a preferred embodiment, the catalyst substrate 23 is a mesh. In a preferred embodiment, the catalyst substrate 23 mesh is in a cylindrical shape. In another preferred embodiment, the catalyst substrate 23 is a corrugated surface that is coated with catalyst and rolled into a spiral shape. In another preferred embodiment, the catalyst substrate 23 is a 3-D honeycomb mesh or one that contains many irregular opening that are impregnated with catalyst. In another preferred embodiment, the catalyst substrate 23 is sponge-like and contains many openings that are impregnated with catalyst. In a preferred embodiment, the catalyst substrate 23 is coated with the catalyst comprising, but not limited to, metals such as palladium, platinum, rhodium or an oxide of the rare earth metal cerium (ceric oxide).

Catalytic combustion is the oxidation of combustibles on a catalytic surface accompanied by the release of heat but without flame. Oxidation of hydrocarbons (HC) to carbon dioxide and water: CxH2x+2+[(3x+1)/2]O2→xCO2+(x+1)H2O. The system favors oxidizing reactions (oxidation of CO and hydrocarbons) when there is an excess of oxygen.

Catalytic combustion is described at least in the following books: Introduction to Catalytic Combustion, R. E. Hayes and Stan T. Kolaczkowski, 1997, Taylor & Francis, Inc., New York, Dalla Betta, Academic Press, ISBN 90-5699-092-6; Combustion: Physical and Chemical Fundamentals, Modeling and Simulation, Experiments, Pollutant Formation 4rd Edition, J. Warnatz, U. Maas, R. W. Dibble; 2006, Springer-Verlag, Berlin, Heidelberg and New York, ISBN 3-540-25992-9; Introduction to Chemical Reactor Analysis, R. E. Hayes, Publisher: CRC Press, 2001, ISBN-10: 1560329262.

By way of example, but not limitation, the catalyst substrate 23 can be in the shape of a ceramic honeycomb, a stainless steel foil honeycomb, a screen made out of steel, or a monolithic honeycomb of aluminum oxide. In many embodiments, these shapes increase the amount of surface area or are coated with additional materials that enhance surface are available to support the catalyst substrate 23. In some embodiments, a wash coat, often as a mixture of silica and alumina, are added to the core and form a rough, irregular surface, which has more surface area and therefore more places for active precious metal sites. In some embodiments, the catalyst, often a precious metal, is added to the washcoat, in suspension, before being applied to the core. Platinum is an active catalyst and is widely used. It is not suitable for all applications, however, because of unwanted additional reactions and/or cost. Palladium and rhodium are two other precious metals used. Platinum and palladium are commonly used as an oxidization catalyst. Cerium, iron, manganese, copper and nickel can also used, although each has its limitations.

The catalytic combustion reaction is initiated following preheating of the catalyst substrate 23 by an activation system 27. In a preferred embodiment, the activation system 27 comprises a resistance based, nichrome wire type activation system which operates off a battery system 29 comprising one or more batteries arranged in series that also powers other systems in the device. In an alternative embodiment, the activation system 27 operates off a dedicated battery system 29 comprising one or more batteries arranged in series. Alternative embodiments may utilize a second battery system. The battery system 29 is capable of supplying sufficient current to operate the fan-blower 19 and the activation system 27, as well as a control system. Examples of batteries that can be used in the battery system 29 include, but are not limited to, lithium metal, lithium polymer and lithium manganese oxide batteries (such as 2CR5).

The activation system 27 having brought the catalytic substrate 23 to the required activation temperature is turned off when the gaseous fuel-air mixture enters and interacts with the warmed catalyst substrate 23 and catalytic combustion reaction is initiated. The heat generated by this catalytic combustion reaction then maintains the catalyst substrate 23 at a temperature above that required for catalytic combustion and the reaction is self propagating. The reaction is shut down by shutting down the fuel supply using either the manually actuated master cutoff valve knob 13 to close the master cutoff valve 15 or through the closure of a proportional valve 47. The proportional valve 47 closes when the latching power switch is returned to the off position and would also shut down if the air temperature feedback signal continues to increase, for instance if air flow is blocked.

In preferred embodiments, once the manually actuated master cutoff valve knob 13 has been turned to the on position from the off position and the latching power switch 11 has been turned to the on position from the off position, ambient air is drawn in by the fan-blower 19 through the air inlet port 3 and the filter 17 which then blows the air through a cowling 31 that connects the fan-blower 19 to a generally cylindrical tube 33 within which lies a heat exchanger 35 through which is in thermal contact with the combustion chamber tube 25. A thermodynamic system is said to be in thermal contact with another system if it can exchange energy with it through the process of heat. In a preferred embodiment, the combustion chamber tube 25 is integral to the heat exchanger 35. In a preferred embodiment, the air inlet port 3 is square such that a square filter 17 can be placed exterior of a fan-blower 19.

In a preferred embodiment, the fan-blower 19 is a centrifugal style fan-blower. In a preferred embodiment, the generally cylindrical tube 33 has an internal cylindrical volume. In a preferred embodiment, this generally cylindrical tube 33 is thermal resistant, such as but not limited to, for example, a hose that is resistant to melting or ignition should a failure occur.

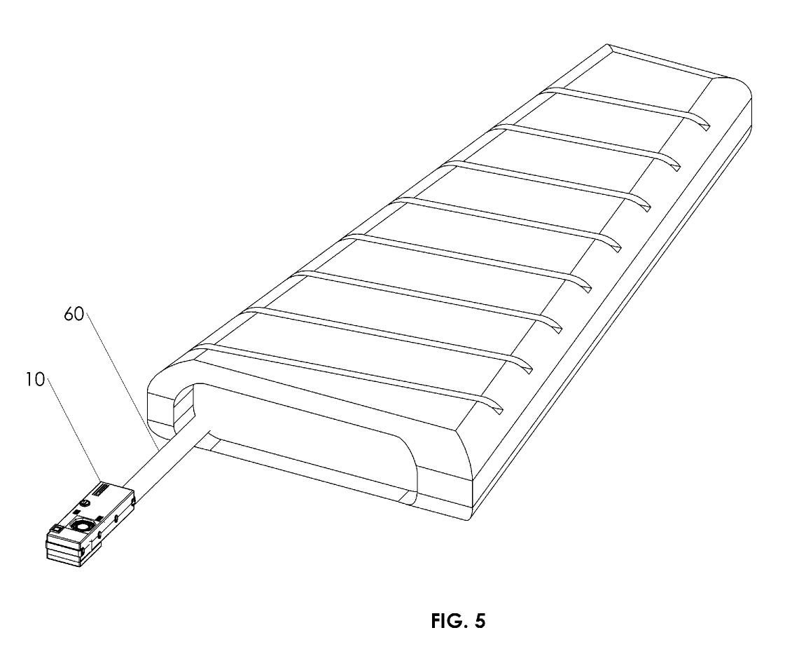

Both conductive and convective forces transfer the heat from the interior combustion chamber tube 25 through the heat exchanger 35 which warms the passing filtered air, and the warmed therapeutic air stream exits the device and is directed at the patient. In preferred embodiments, the air enters tubing for delivery to the patient. In some embodiments the tubing is flexible. In some embodiments, the tubing has a diffuser of its end. In preferred embodiments, the warmed air is directed into a thermally efficient container such as, but not limited to, blankets, sheets, tarps and alike. In another preferred embodiment, the warmed air is directed into a thermally efficient container such as, but not limited to, a sleeping bag (as shown in FIG. 5), a body bag (cadaver pouch), a box or alternatively it can be connected to one of the devices or garments previously described as thermal blankets, (U.S. Pat. Nos. 4,572,188; 5,405,371; 6,524,332), inflatable covers (U.S. Pat. No. 7,578,837) and clinical garments (WO 2003/086500; US2006/0122671, US2007/0093885) in which the patient at risk of hypothermia is placed.

In a preferred embodiment, the heat exchanger 35 further comprises a multiplicity of heat transfer protrusions 37 affixed to the combustion chamber tube 25. In one preferred embodiment, the heat transfer protrusions 37 are fins. In another preferred embodiment, the heat transfer protrusions 37 are ring like disks. In another preferred embodiment, the invention further comprises a thermally conductive metallic mesh located between the combustion chamber tube 25 and within the internal cylindrical volume of the generally cylindrical tube 33. In another preferred embodiment, the invention further comprises a thermally conductive metallic sponge like material located between the combustion chamber tube 25 and within the internal cylindrical volume of the generally cylindrical tube 33. Examples of thermally conductive metallic materials include but are not limited to aluminum, copper and gold.

In another embodiment, the heat exchanger 35 further comprises a flexible heat pipe 60 to transfer heat from the combustion chamber 25 to the therapeutic air stream. A heat pipe is a heat transfer mechanism that combines the principles of both thermal conductivity and phase transition to efficiently manage the transfer of heat between two solid interfaces. Heat pipes are closed systems that exploit the energy required to vaporize and condense a working fluid to maximize heat transfer. Copper is often the material of choice when constructing a heat pipe, with a solid sealing wall on the exterior, a porous internal wall and hollow center. A heat pipe commonly uses ethanol as the working fluid. The heat from the combustion reaction vaporizes the working fluid at the “hot” end, with the vapor moving to the center of the heat pipe. The hot vapor then travels to the “cold” end where it condenses and migrates back through the hot end via capillary action through the porous wall.

In a preferred embodiment, the heat exchanger 35 can be assembled using distinct parts, for example, heat transfer protrusions 37, a combustion chamber tube 25 and a generally cylindrical tube 33. In an alternative embodiment, the heat exchanger 35 can be machined out of, for example, a single cylinder of aluminum, copper or gold, in which the combustion chamber tube is created, for example, by drilling out of the center of the cylinder.

FIG. 6 is an exploded view of another embodiment of a portable body warming device according to the present invention. The device includes a housing 1 exhaust vent 9 and intake filter 17 as previously discussed. A fuel line 55 connects a fuel tank 39 to the combustion chamber 25 and the heat exchanger 35. The control board 45 and battery system 29 have been relocated from the FIG. 1 embodiment and the master cutoff valve 15 and the latching power switch 11 have been repositioned in the FIG. 6 embodiment. A proportional valve 47 is shown, and serves the same previously discussed function as well as the generally cylindrical tube 33. Although not shown in FIG. 6, the assembly includes an activation system 27, as previously disclosed.

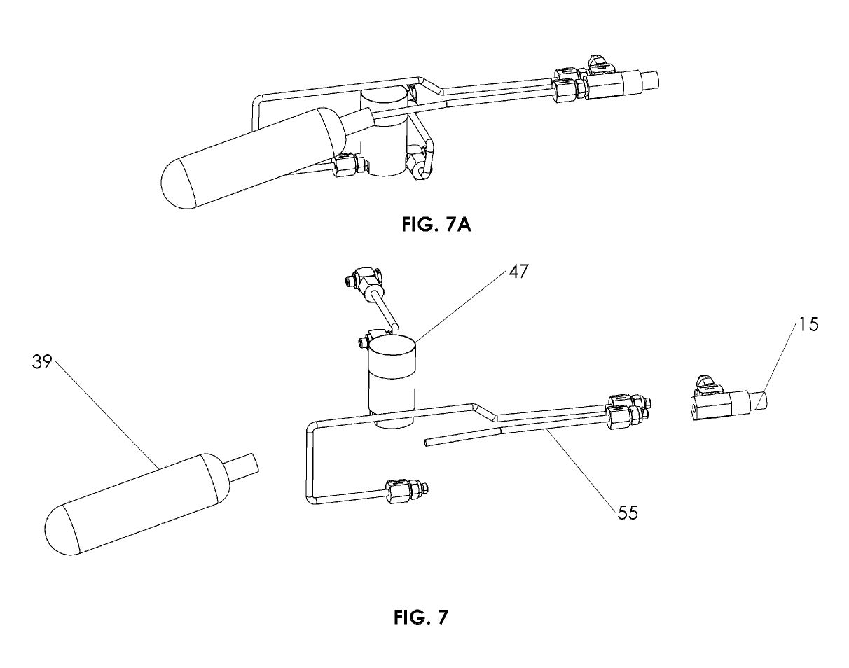

FIG. 7 shows an exploded view of some of the components of the fuel system discussed above, including a fuel tank 39, a master cutoff valve 15 and a proportional valve 47 are shown. Conventional fuel line 55 tubes and connections interconnect these components in a fluid type manner, thereby producing the assembly as shown in FIG. 7A.

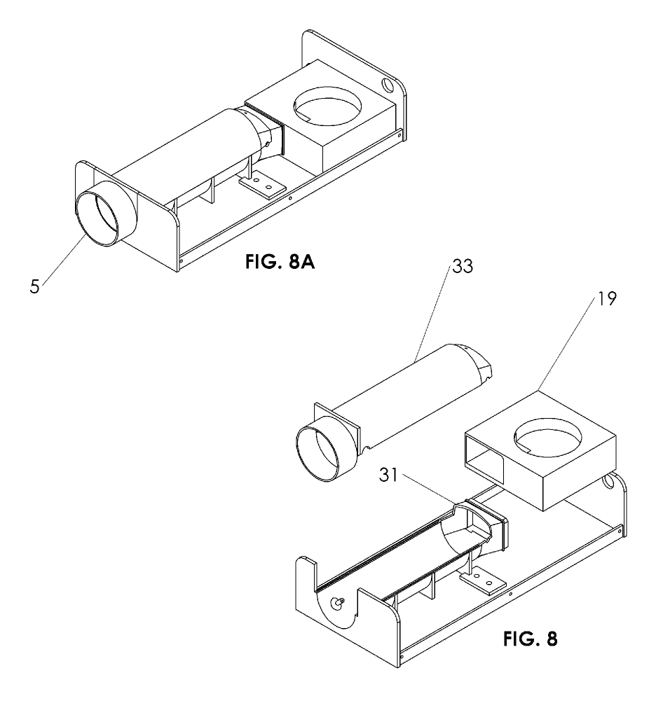

FIG. 8 shows the generally cylindrical tube 33 of this modified embodiment, with the right-hand end of tube 33 adapted for positioning within the column. Fan-blower 19 serves a purpose as previously disclosed. FIG. 8A shows the components of FIG. 8 assembled.

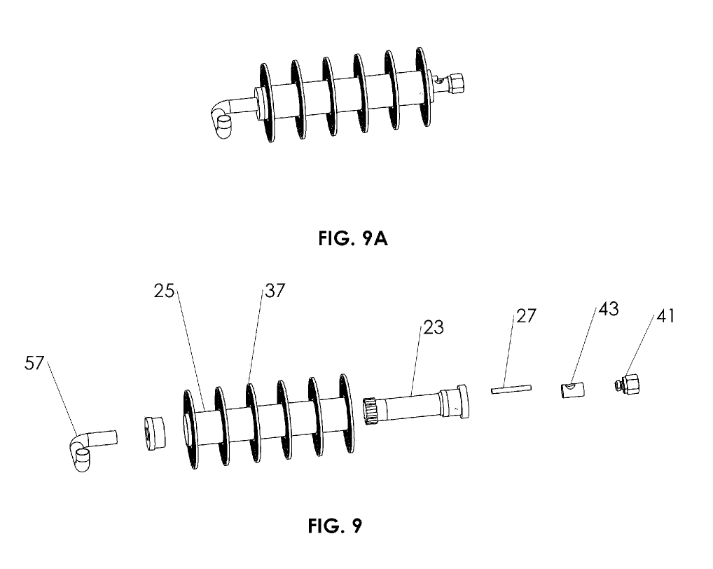

FIG. 9 shows an exploded view of some of the components of the heat exchange system discussed above, including fuel orifice 41, venturi ports 43, activation system 27 and catalytic substrate 23 which lies within a combustion chamber tube 25 having heat transfer protrusions 37, that is connected to an exhaust tube 57, thereby producing the assembly as shown in FIG. 9A.

A preferred embodiment further comprises a source of combustible gaseous hydrocarbon in a fuel tank 39 in fluid communication with the proximal end of the gas delivery line. In a preferred embodiment, the gaseous hydrocarbon is selected from the group consisting of methane, ethane, propane, and butane. One embodiment further comprises a rectangular shape to the fuel tank 39, which maximizes the use of space. In an alternate embodiment, the fuel tank may have a generally rectangular shape. Also, the proximal end of the gas delivery line may connect to the top of the fuel tank 39 to take advantage of the rising nature of the fuel gases. In some embodiments, the gas delivery line comprises a distal end region where a fuel orifice 41 in the cowling 31 creates a high velocity jet which provides an air entrainment system utilizing venturi ports 43. In one embodiment, the use of venture ports 43 draws air to mix with the fuel from the fuel orifice 41. In the alternative embodiment, shown in FIG. 6, the blower forces the fuel into the combustion chamber combustion chamber tube 25 and thus the need for venturi ports 43 is eliminated and the fuel orifice 41 may be much smaller.

In some embodiments, the proximal end of the gas delivery line connects to the top of the fuel tank 39 to take advantage of the rising nature of the fuel gases created.

In preferred embodiments, the gas delivery line further comprises a master cutoff valve 15 which is connected to the manually actuated master cutoff valve knob 13. In a preferred embodiment, the master cutoff valve 15 is a needle valve, although those of skill in the art can appreciate the potential use of other valve types, although one type of a ball valve did not suffice as it had a tendency to leak. In a preferred embodiment, a gas delivery line is further connected to a pressure switch 21 which closes the circuit between the positive terminal of a battery system 29 and a control board 45. In preferred embodiments, the control board 45 contains a microcontroller. In preferred embodiments, upon powering up the control board 45, the microcontroller goes into a passive mode, conserving battery life and restricting gas flow while waiting for the latching power switch 11 to be pushed to initiate activation of combustion. Preferably, the master cutoff valve 15 would be opened just prior to pressing the latching power switch 11. The latching power switch 11 prevents unwanted startup and also allows the operator to shut down the device in a way that cools the heat exchanger 35 before the fan is turned off. In preferred embodiments, the opening of the master cutoff valve 15 produces a combustible gaseous fuel within the combustion chamber tube 25 at the catalyst substrate 23.

In preferred embodiments the fuel line 55 further comprises a proportional valve 47 which opens and closes proportionally in response to a change in a voltage signal, thus providing a more precise means of regulating fuel flow.

In one embodiment, the gas delivery line is connected to a fuel filter 51 to remove any particulates in the fuel line 55. The distal end of the gas delivery line connects with a fuel orifice 41 in the cowling 31 that creates a high velocity jet which provides an air entrainment system utilizing venturi ports 43. This produces a combustible gaseous fuel-air mixture within the combustion chamber tube 25. In a preferred embodiment, the combustion chamber tube 25 approximates an L-shape with an axial leg portion and a radial leg portion. In another embodiment, the combustion chamber tube 25 is connected to an exhaust tube 57.

In some embodiments, after passing through the heat exchanger 35, the distal portion of the combustion chamber tube 25 bends such that it passes close enough to the fuel tank 39 such that residual heat warms the liquid fuel in the fuel tank 39 to help warm the fuel and maintain vapor pressure.

In some preferred embodiments, the radial leg portion of a L-shaped combustion chamber tube 25 crosses over the top of the fuel tank 39 distal from the gas delivery line connection. In other preferred embodiments, the combustion chamber tube 25 transfers heat to the fuel tank 39 using a heat sink. In other preferred embodiments, the combustion chamber tube 25 is designed so that the residual combustive gases are released in close proximity to the fuel tank 39. In preferred embodiments, the combustive gases are then released passively through the venting 19 at the top of the outer housing 1.

Another embodiment of the present system comprises process controls for controlling the temperature of the warm air output from the portable body warming device. In a preferred embodiment, the invention further comprises a controller 57 operatively connected to receive the temperature signal from the sensor and transmit a control signal responsive to the temperature signal. In one preferred embodiment, the controller is a microcontroller. In other embodiments the controller can be, but is not limited to a Field Programmable Gate Array (FPGA) or Application Specific Integrated Circuit (ASICs). In a preferred embodiment, the invention controller is a microcontroller.

In another embodiment, the controller is an analog controller. In a preferred embodiment, when the temperature signal indicates that the temperature of the air passing through the outlet section of tube exceeds a preselected temperature threshold, a control signal to increase the degree of closure of the proportional valve 47 is generated. In a preferred embodiment, the proportional valve 47 is coupled to receive the control signal from the controller. In other embodiments, temperature may be regulated by controlling fuel flow to the catalyst substrate 23. If the sensed temperature is below a certain value the controller sends a signal to the proportional valve 47 and increases gas flow to the catalytic combustion chamber.

In a preferred embodiment, when the temperature signal indicates that the temperature of the warmed therapeutic air stream passing through the outlet section of flow channel exceeds a preselected temperature threshold, a control signal is sent to adjust the degree to which the proportional valve 47 is open.

In another embodiment, a manually operated damper for controlling the flow rate of air between the inlet port 3 and the discharge port 5 can be adjusted to regulate air flow through the heat exchanger 35. When the temperature of the warmed therapeutic air stream passing through the outlet section of the air flow path deviates from a preselected temperature threshold, a control signal is sent to open or close the damper to increase or decrease the air flow over the heat exchanger 35.

An embodiment of the presently described portable body warming device further comprises a control system that includes software programmed into a control board 45 that contains a microcontroller. In some embodiments, the control board 45 is housed within the outer housing 1. In an alternative embodiment, the control board 45 is housed within a tray 59 which is attached to the outer housing 1. This control system monitors and actuates several components of the apparatus. In preferred embodiments, this invention further comprises inputs to the control system to monitor the temperature of the air flowing from the outlet section and the temperature of the combustion chamber tube 25. In a preferred embodiment, the temperature sensor is selected from the group consisting of a thermistor, a thermocouple, and a solid state thermal sensor. In a preferred embodiment, the temperature sensor used to monitor the outlet air temperature is a thermistor and the sensor used to monitor the temperature of the combustion chamber 25 is a thermocouple.

In a preferred embodiment, the temperature setting for the control system monitoring the temperature of the air flowing from the outlet section is preselected and fixed. This prevents accidental overheating of patients and provides an additional safety feature that negates potential operator error, or accidental adjustments, for example, during transport. In an alternative embodiment, the temperature setting for the control system monitoring the temperature of the air flowing from the outlet section can be varied by the operator by adjusting the set point of the control system.

In preferred embodiments, monitoring the temperature of the combustion chamber tube 25 also verifies that the catalyst substrate 23 is lit, directs the regulation of fuel flow through the proportional valve 47, and directs the microcontroller to regulate the fan-blower 19. Thus, the control system program can attempt re-activation of combustion should a “flameout” type of condition occur. In a preferred embodiment, this information is used by the microcontroller to regulate fuel flow via the proportional valve 47, activation of combustion via an activation system 27 and the activity of the fan-blower 19. In preferred embodiments, the control system can also direct the device to perform a shutdown operation to cool the heat exchanger 35 after the latching power switch 11 has been switched to the off position from the on position. The shutdown sequence closes the proportional valve 47 and keeps the fan-blower 19 running for a short period of time to cool the heat exchanger 35 to prevent overheating inside the device.

In preferred embodiments, the centrifugal style fan-blower 19, a microcontroller, and the proportional valve 47 are powered by a lithium metal battery system 29. In some embodiments, the air warming system may be used for a limited time (mission, alpine ascent or race) and once consumed, rather than slowing the user down by having to carry it out—the device can be disposed of. Alternatively, in other embodiments, the presently described portable body warming device could easily be adapted for repeat usage. These adaptations include but are not limited to a fuel tank 39 that can be removed and replaced easily, using an adapter. Such adapters are known, but not limited to, those used for small canisters of CO₂ in air guns.

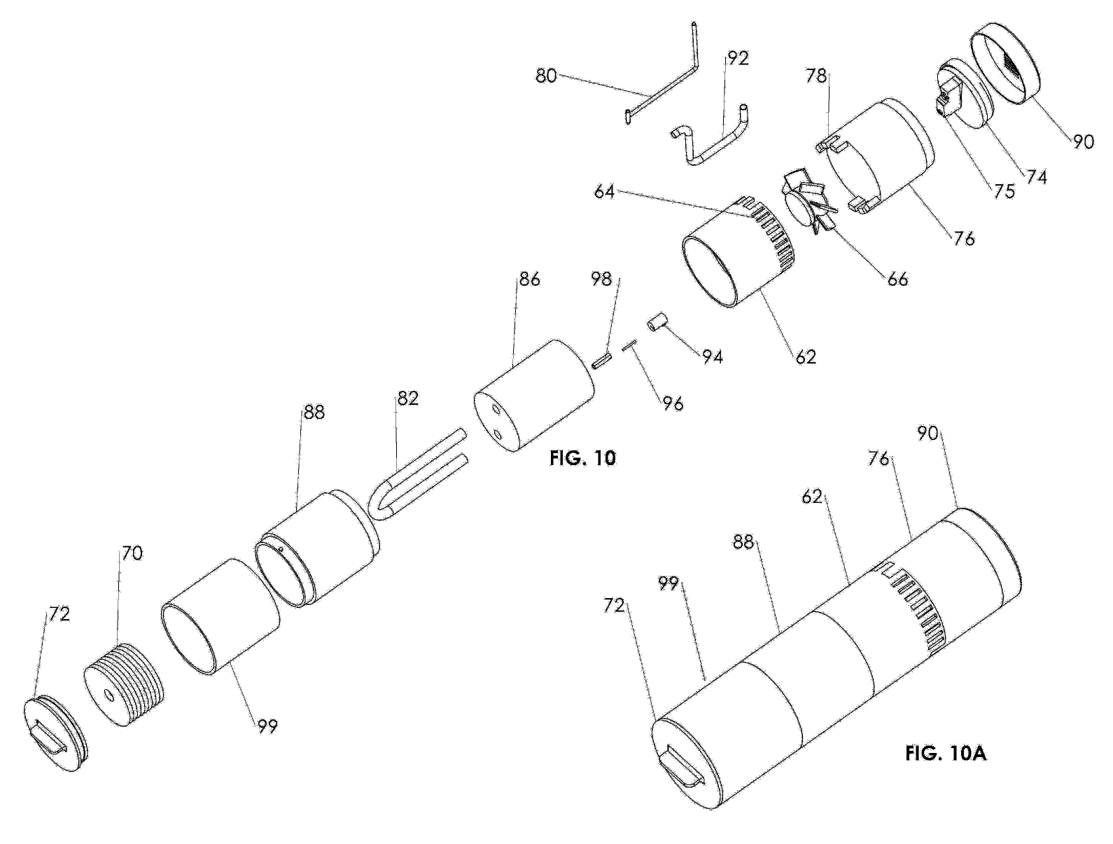

FIG. 10 is an exploded view of a more compact version of a portable body warmer according to this invention. Air enters the fan housing 62 through circumferential slots 64. Fan housing 62 encloses a powered fan 66 which moves air through the heat exchanger housing 88 and then through the interior of the hose 70, which is shown in its collapsed position in FIG. 10. Hose housing 99 may be blocked by end cap 72. A reduced size fuel tank 74 and control valve 75 are provided at the opposite end of tubular housing 76, which has a plurality of axially extending tabs 78 for engagement with respective slots in the fan housing 62. Fuel tank line 80 interconnects the fuel tank 74 with the U-shaped tube 82. Air from the fan enters the combustion chamber within housing 62, which is in thermal communication with heat exchanger 86. U-tube 82 is positioned substantially within the heat exchanger 86. The discharge from the U-tube 82 enters the combustion chamber and is pushed by the fan 66 back to hose 70 and through the heat exchanger 86 which is within tubular housing 88.

FIG. 10 also illustrates end cap 90 for enclosing the fuel tank 74, and igniter housing 94 housing igniter 96 and catalyst 98. Exhaust line 92 is fluidly connected to the U-tube 82, and serves to discharge combustion gases to the rear of the warmer and out the back cap 90. Heat from the combustion exhaust is used to warm the fuel tank, which normally cools as fuel is consumed. FIG. 10 illustrates the compact body warmer in the assembled position.

Air may optionally be filtered before entering housing 62. A fuel cut-off valve knob, a combustion reaction system, batteries, a proportional valve, a control board, and other components discussed above may also be used in the FIG. 10 embodiment, but are not shown for clarity of the alternate components shown.

The present invention is well suited for accomplishing the goal of providing a safe and effective portable body warming system to prevent or treat hypothermia in a patient. The portable body warming device is relatively light and compact, and provides a portable system that can easily be carried into the field and used to treat a patient. The body warming system is not dependent on power that is not included within the warming device, and the device provides for the separation of air used for combustion from the air which is warmed and directed to the patient.

In some embodiments, the patients described as suffering from or at risk for hypothermia are either human or veterinary, such as but not limited to, dogs used to pull sleds for transportation and racing in cold climates.

Without further elaboration, it is believed that one skilled in the art can, using the description herein, utilize the present invention to its fullest extent. The foregoing disclosure and description of the invention are illustrative and explanatory. Various changes in the size, shape, and materials, as well as in the details of the illustrative construction may be made without departing from the spirit of the invention.

While the preferred embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A portable body warmer to counteract hypothermia, comprising:
a housing having an air inlet port for receiving ambient air, a warmed air discharge port in fluid communication with the inlet port for discharging therapeutic warmed air, and a exhaust vent port;
a powered blower for moving air through the housing along a substantially closed airflow path from the air inlet port to the discharge port;
a combustion chamber within the housing, the combustion chamber housing a catalyst and being in fluid communication with the exhaust vent port for discharging combustion byproducts from the housing;
an activator for initiating combustion of a gaseous fuel in the combustion chamber;
a heat exchanger within the housing for transferring heat from the combustion chamber to the closed therapeutic airflow path extending between the air inlet port to the discharge port;
a portable hydrocarbon gas fuel source in fluid communication with the combustion chamber; and
a battery for powering the blower and the activator.

2. The portable body warmer as defined in claim 1, further comprising:
a temperature sensor for sensing the temperature of air discharged from the discharge port; and
a controller for regulating the gas flow to the combustion chamber if the sensed temperature exceeds or falls below a selected value.

3. The portable body warmer as defined in claim 2, further comprising:
a proportional valve responsive to the controller for controlling fuel flow to the combustion chamber.

4. The portable body warmer as defined in claim 1, further comprising:
a combustion air inlet port in the housing for passing ambient air to the combustion chamber.

5. The portable body warmer as defined in claim 4, wherein the combustion air inlet port is provided adjacent one end of the housing, and the exhaust vent port is provided adjacent an opposing end of the housing to minimize combustion gases from the combustion chamber entering the housing through the inlet port.

6. The portable body warmer as defined in claim 1, wherein the combustion chamber is defined by an elongate tube housing the catalyst and in thermal contact with a heat exchanger.

7. The portable body warmer as defined in claim 6, wherein the elongate tube is substantially L-shaped, with an axial leg portion of the tube housing the catalyst and the heat exchanger, and a radial leg portion of the tube passing the combustion byproducts radially from the housing.

8. The portable body warmer as defined in claim 7, wherein the air discharge from the radial leg warms the fuel in the portable hydrocarbon gas fuel source.

9. The portable body warmer as defined in claim 1, further comprising:
a manually operated damper for controlling the flow rate of air between the inlet port and the outlet port.

10. A portable body warmer as defined in claim 1, wherein the combustion chamber is defined by a substantially U-shaped tube housing the catalyst.

11. A portable body warmer as defined in claim 10, wherein the U-shaped tube is substantially within the heat exchanger.

12. A portable body warmer to counteract hypothermia, comprising:
a housing having an air inlet port for receiving ambient air and a warmed air discharge port in fluid communication with the inlet port for discharging warmed air;
a powered blower for moving air through the housing along a substantially closed airflow path from the inlet port to the discharge port;
an elongate tube within the housing defining a combustion chamber within the housing, the combustion chamber housing a catalyst and discharging combustion byproducts from the housing through an exhaust vent port;
an activator for initiating combustion of gaseous fuel in the combustion chamber;
a heat exchanger within the housing and in thermal contact with the combustion chamber for transferring heat from to the closed airflow path extended between the inlet port and the discharge port;
a portable hydrocarbon gas fuel source in fluid communication with the combustion chamber; and
a battery for powering the blower and the activator.

13. The portable body warmer as defined in claim 12, further comprising:
a combustion air intake port in the housing for passing ambient air to the combustion chamber; and
an exhaust vent port in the housing for discharging combustion byproduct from the housing.

14. A portable body warmer as defined in claim 12, wherein the elongate tube houses the catalyst and is in thermal contact with the heat exchanger.

15. The portable body warmer as defined in claim 12, wherein the elongate tube is substantially L-shaped, with an axial leg portion of the tube housing the catalyst and in fluid contact with the heat exchanger, and a radial leg portion of the tube passing air radially from the housing.

16. The portable body warmer as defined in claim 12, wherein the combustion chamber is defined by a substantially U-shaped tube housing the catalyst.

17. A method of warming a body to counteract hypothermia, comprising:
providing a portable housing having an air inlet port for receiving ambient air, a warmed air discharge port in fluid communication with the inlet port for discharging warmed air, and an exhaust vent port;
positioning a combustion chamber within the housing, the combustion chamber housing a catalyst and being in fluid communication with the exhaust vent port for discharging combustion byproducts from the housing;
activating the combustion of gaseous fuel in the combustion chamber;
providing a heat exchanger within the housing for transferring heat from the combustion chamber to the closed therapeutic airflow path extending between the inlet port and the discharge port;
providing a portable hydrocarbon gas fuel source in fluid communication with the catalytic combustion chamber; and
moving warmed air through the housing along a substantially closed airflow path extending from the air inlet port to the air discharge port and transmitting the warmed air to counteract hypothermia.

18. The method as defined in claim 17, further comprising:
transmitting the warmed air from the housing through a flexible tube to a covering over at least a portion of a patient.

19. The method as defined in claim 17, wherein the catalytic combustion chamber is defined by an elongate tube housing the catalyst and in fluid contact with the heat exchanger.

20. The method as defined in claim 17, wherein the combustion reaction involves a catalyst confined within the combustion chamber.
